(19) United States
(12) Patent Application Publication
(10) Pub. No.: US 2026/0175046 A1
(43) Pub. Date: Jun. 25, 2026

(54) METHOD AND APPARATUS FOR OPTIMIZING BEAM ANGLE OF PARTICLE THERAPY

(71) Applicant: SAMSUNG LIFE PUBLIC WELFARE FOUNDATION, Seoul (KR)

(72) Inventors: Sang Hee AHN, Seoul (KR); Hee Chul PARK, Seoul (KR); Jeong II YU, Seoul (KR)

(73) Assignee: SAMSUNG LIFE PUBLIC WELFARE FOUNDATION, Seoul (KR)

(21) Appl. No.: 19/414,840

(22) Filed: Dec. 10, 2025

Related U.S. Application Data
(63) Continuation of application No. PCT/KR2024/013044, filed on Aug. 30, 2024.

(30) Foreign Application Priority Data
Aug. 30, 2023 (KR) 10-2023-0114403

(51) Int. Cl. A61N 5/10; G16H 20/40

(52) U.S. Cl. CPC A61N 5/1031; A61N 5/1037; A61N 5/1039; G16H 20/40; A61N 2005/1087

(57) ABSTRACT

A beam angle optimization method for particle therapy comprises: receiving, by an analysis apparatus, distance information from a patient's body surface to a tumor, internal heterogeneity information, internal transmission information of a particle beam, and internal motion information according to the patient's respiration; inputting, by the analysis apparatus, the distance information from the patient's body surface to the tumor, the internal heterogeneity information, the internal transmission information of the particle beam, and the internal motion information according to the patient's respiration into a deep learning model; and calculating, by the analysis apparatus, a particle therapy beam angle based on an output value of the deep learning model.

### CROSS-REFERENCE TO RELATED APPLICATION

This application claims the benefit of priority under 35 U.S.C. § 119(a) to Korean Patent Application No. 10-2023-0114403, filed Aug. 30, 2023, and is a continuation under 35 U.S.C. § 365(c) of International Application No. PCT/KR2024/013044, filed Aug. 30, 2024, the entire disclosures of which are incorporated herein by reference in their entireties.

### BACKGROUND

### 1. Field

The present disclosure relates to a method and an apparatus for optimizing a particle beam angle during particle therapy by utilizing deep learning technology.

### 2. Discussion of Related Art

Radiation therapy is a treatment that destroys tumors by using radiation such as X-rays, gamma rays, electron beams, or particle beams. Among radiation therapies, particle therapy is a treatment method that destroys cancer tissues by using particles. Particle therapy is a treatment method based on the Bragg Peak principle, which can destroy a tumor while minimizing damage to normal tissues by using particles. The Bragg Peak principle refers to a phenomenon in which a particle beam emits an enormous amount of radiation energy at the moment when it passes through normal tissue and reaches the tumor.

### SUMMARY

In particle therapy, due to the characteristics of the Bragg Peak, changes in internal organs can greatly affect the treatment. Therefore, before performing particle therapy, it is necessary to establish an optimal treatment plan so that the therapeutic radiation can be delivered to the tumor as planned while considering organ movement. Specifically, when performing particle therapy, a treatment plan must be established to minimize damage to critical organs around the tumor caused by radiation while allowing the maximum amount of radiation to be delivered to the tumor region.

In particular, determining the angle of a particle beam when establishing a treatment plan is very important in particle therapy. For example, when a particle beam passes through a path containing many heterogeneous materials, its range can change, increasing the likelihood that the beam will not be delivered as intended.

This also holds when an organ subject to respiratory motion lies along the beam path.

In such a case, the particle beam may be delivered to a normal organ instead of the tumor or may fail to deliver a sufficient dose to the tumor, which may cause the recurrence of cancer in the treated area. However, determining the particle beam angle requires highly advanced clinical experience and knowledge, and there has been a problem in that it takes a long time to determine the beam angle.

The present disclosure aims to provide a method that optimizes the particle beam angle during particle therapy by utilizing deep learning technology.

### Technical Solution

A beam angle optimization method for particle therapy, comprising: receiving, by an analysis apparatus, distance information from a patient's body surface to a tumor, internal heterogeneity information, internal transmission information of a particle beam, and internal motion information according to the patient's respiration; inputting, by the analysis apparatus, the distance information from the patient's body surface to the tumor, the internal heterogeneity information, the internal transmission information of the particle beam, and the internal motion information according to the patient's respiration into a deep learning model; and calculating, by the analysis apparatus, a particle therapy beam angle based on an output value of the deep learning model.

By using the technology described below, the time consumed in establishing a particle therapy plan can be reduced while improving the quality of the particle therapy plan. Through this process, it is possible to reduce the waiting time for patients receiving particle therapy or to increase the number of patients who can receive particle therapy.

### BRIEF DESCRIPTION OF THE DRAWINGS

FIG. 1 illustrates an overall process in which an analysis apparatus (100) optimizes a particle beam angle.

FIG. 2 shows one embodiment (200) of a particle beam angle optimization method.

FIG. 3 illustrates one embodiment of distance information from a patient's body surface to a tumor and internal heterogeneity information.

FIG. 4 shows one embodiment of internal motion information.

FIG. 5 illustrates one embodiment of a process of a patient-specific beam angle calculation technique for particle therapy.

FIG. 6 shows one embodiment for calculating a therapeutic effect according to a particle beam treatment angle.

FIG. 7 shows another embodiment in which an analysis apparatus calculates a particle therapy beam angle.

FIG. 8 shows one embodiment of the configuration of an analysis apparatus (300).

FIG. 9 shows one embodiment of a deep learning model.

### DETAILED DESCRIPTION

The technology described below can be modified in various ways and may have various embodiments. Specific embodiments of the technology described below may be described in the drawings of the specification. However, this is for describing the technology described below, and is not intended to limit the technology described below to specific embodiments. Accordingly, it should be understood that all modifications, equivalents, or alternatives included in the spirit and technical scope of the technology described below are included in the technology described below.

In the terms used below, unless clearly interpreted otherwise in context, singular expressions should be understood to include plural expressions, and terms such as “comprise” should be understood as indicating the presence of listed features, numbers, steps, operations, components, parts, or combinations thereof, rather than excluding the presence or addition of one or more other features, numbers, steps, operations, components, parts, or combinations thereof.

Before describing the drawings in detail, it is intended to clarify that the classification of components in this specification is merely a classification according to the main functions performed by each component. That is, two or more components to be described below may be combined into one component, or one component may be divided into two or more components according to more segmented functions and provided. In addition, each of the components to be described below may additionally perform some or all of the functions among the functions performed by other components, and some of the functions among the main functions performed by each component may be performed exclusively by another component.

In performing a method or an operation method, unless a specific order is clearly described in context for each process constituting the method, the processes may occur in an order different from the stated order. That is, the processes may occur in the same order as stated, may be performed substantially simultaneously, or may be performed in the reverse order.

In the technology described below, particle therapy may be a treatment method used to treat cancer with particles. Particle therapy may be a treatment method that destroys cancer cells by using high-energy particles. In one embodiment, particle therapy may include proton therapy and/or heavy-ion therapy.

The technology described below relates to a patient-specific beam angle calculation technique for particle therapy.

Hereinafter, it is described that an analysis apparatus optimizes a beam angle by using a learning model. The analysis apparatus may be implemented as various devices capable of data processing. For example, the analysis apparatus may be implemented as a PC, a server on a network, a smart device, or a chipset in which a dedicated program is embedded.

Hereinafter, an overall process in which the analysis apparatus performs a method of optimizing a particle beam angle is described.

As used herein, the term ‘treatment-planning score’ refers to a quantitative evaluation value computed from predicted dose distribution parameters, such as PTV coverage or OAR dose penalty, as a function of gantry angle (0°-360°)

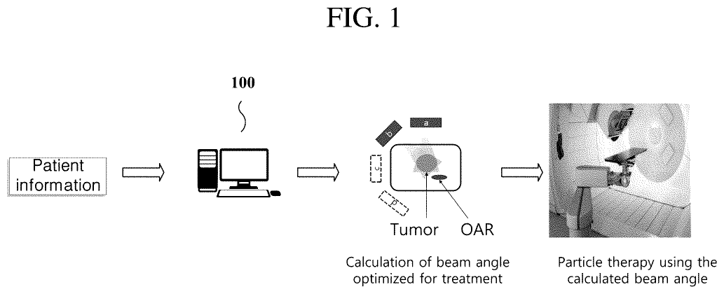

FIG. 1 illustrates an overall process in which an analysis apparatus (100) optimizes a beam angle for particle therapy.

The analysis apparatus may receive distance information from a patient's body surface to a tumor, internal heterogeneity information, internal transmission information of a particle beam, and internal motion information according to the patient's respiration. The analysis apparatus may input the distance information to the tumor, the internal heterogeneity information, the internal transmission information of the particle beam, and the internal motion information according to the patient's respiration into a deep learning model. The analysis apparatus may calculate a particle therapy beam angle based on an output value of the deep learning model. Particle therapy may be performed based on the particle therapy beam angle thus calculated.

Hereinafter, a method of optimizing a particle beam angle is described in detail.

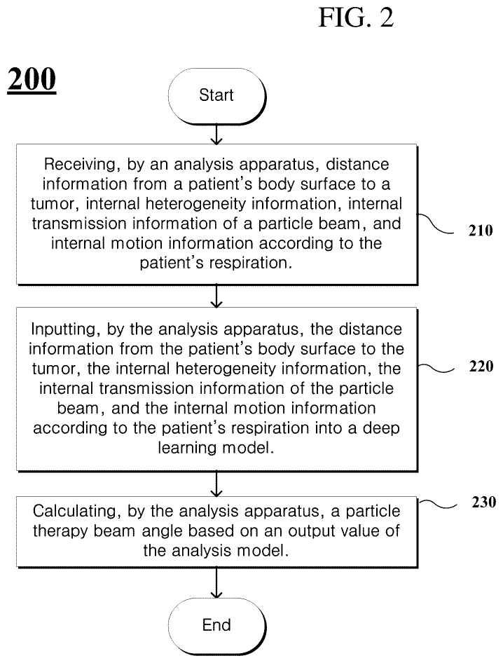

FIG. 2 is one embodiment (200) of a particle beam angle optimization method.

The analysis apparatus may receive distance information from a patient's body surface to a tumor, internal heterogeneity information, internal transmission information of a particle beam, and internal motion information according to the patient's respiration (210).

The distance information from the patient's body surface to the tumor may mean a distance (radiological depth) through which the particle beam passes from the patient surface to the tumor. That is, the distance information from the patient's body surface to the tumor may mean how far the tumor is from the patient's surface.

The internal heterogeneity information may include information on how homogeneous the interior of the body through which the particle beam passes is. Specifically, the internal heterogeneity information may be information calculated based on a standard deviation value of HU (Hounsfield Unit) acquired from a CT image.

The internal transmission information of the particle beam may mean how well the particle beam passes through the body. Specifically, the internal transmission information of the particle beam may mean information on how much a critical organ in the body transmits the particle beam. For example, the internal transmission information of the particle beam may include a penetration weight of OAR (Organs at Risk) indicating a degree to which the particle beam passes through a critical organ in the body.

The internal motion information according to the patient's respiration may mean information on a degree to which internal organs change as the patient breathes. Specifically, the internal motion information according to the patient's respiration may include information calculated from DVF (deformation vector fields). This may be acquired from 4D CT containing the patient's respiration information. In one embodiment, the internal motion information may include DVF values calculated from 4D CT at breathing phases 0 to 90. Alternatively, the internal motion information may include DVF values calculated from 4D CT at breathing phases 0 to 50. Alternatively, the internal motion information may include DVF values calculated from 4D CT at breathing phases 50 to 90.

The distance information from the patient's body surface to the tumor, the internal heterogeneity information, the internal transmission information of the particle beam, and the internal motion information according to the patient's respiration may be information acquired from a CT (Computed Tomography) image. Specifically, the information may be acquired from 4D CT images reflecting the patient's respiratory motion.

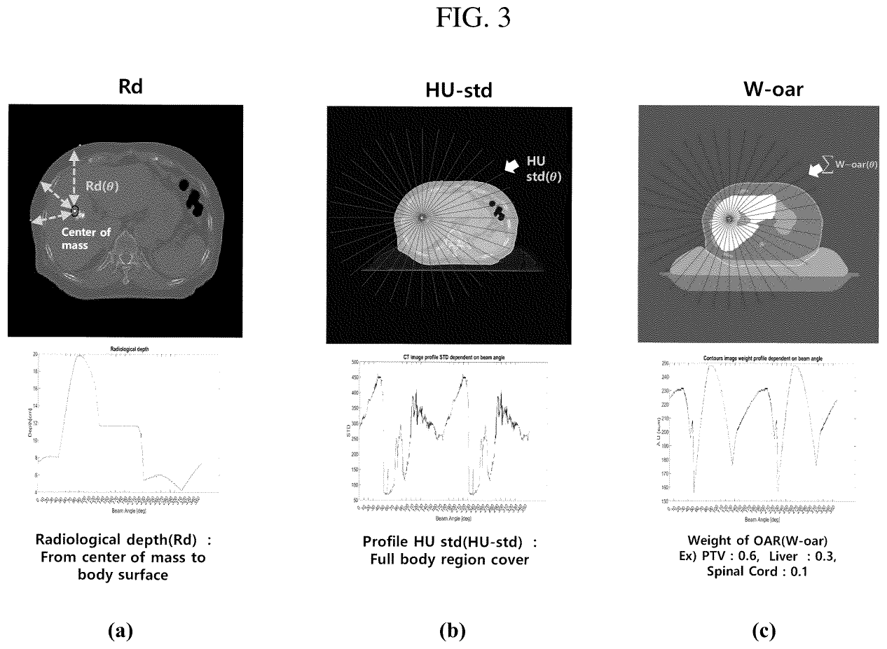

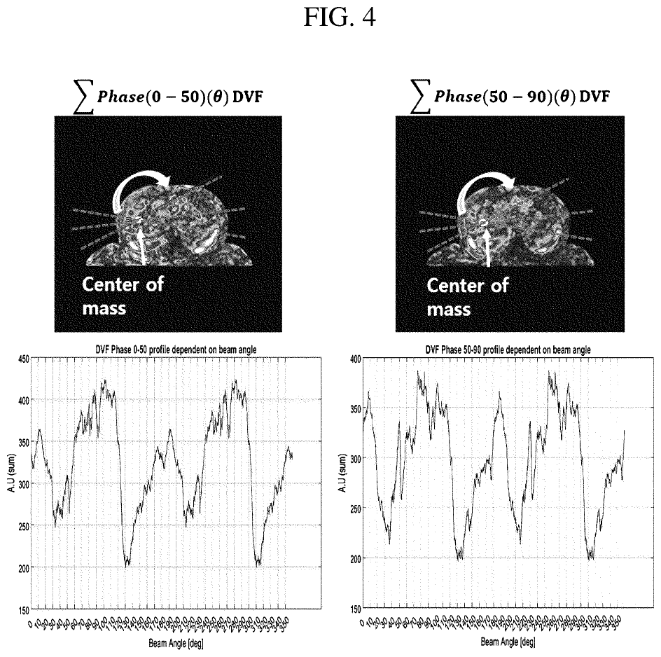

The distance information from the patient's body surface to the tumor, the internal heterogeneity information, the internal transmission information of the particle beam, and the internal motion information according to the patient's respiration may be information according to a particle beam angle. For example, as shown in FIGS. 3 and 4, the distance information from the patient's body surface to the tumor, the internal heterogeneity information, the internal transmission information of the particle beam, and the internal motion information according to the patient's respiration may have a form of graphs. FIG. 3(a) shows the distance information from the body surface to the tumor. FIG. 3(b) shows the internal heterogeneity information. FIG. 3(c) shows the internal transmission information of the particle beam. FIG. 4 shows the internal motion information according to respiration.

The analysis apparatus may input the distance information from the patient's body surface to the tumor, the internal heterogeneity information, the internal transmission information of the particle beam, and the internal motion information according to the patient's respiration into a deep learning model (220).

The deep learning model may be a model trained to output a particle therapy beam angle based on training data.

In one embodiment, the deep learning model may include a model based on an artificial neural network. For example, the deep learning model may include CNN (Convolutional Neural Network), RNN (Recurrent Neural Network), RBM (Restricted Boltzmann Machine), DBN (Deep Belief Network), and GAN (Generative Adversarial Network), RL (Reinforcement Learning), and a Transformer Neural Network.

The analysis apparatus may calculate a particle therapy beam angle based on an output value of the deep learning model (230).

In one embodiment, the analysis apparatus may calculate a particle therapy beam angle that is determined to be most suitable for performing particle therapy among 360 degrees. For example, the analysis apparatus may derive, in the form of a peak or a specific region of a predicted graph, a portion that is evaluated to have the best therapeutic effect among 360 degrees.

The following describes one embodiment for constructing a deep learning model.

First, data of patients who have received particle therapy may be collected. The patient data may include 4D CT images containing the patient's respiratory information.

The data of patients who have received particle therapy may be processed.

In one embodiment, heterogeneous region information of patient tissue may be extracted from CT images. The heterogeneous region information may include Air, Bone, Metal material (Gold fiducial, stent), and Tissue, etc. Specifically, it may be possible to distinguish regions where organs exist or regions where tumors exist from CT images. For this purpose, a segmentation model may be used. The segmentation model may be a trained learning model trained to segment heterogeneous regions in CT images or to distinguish regions where tumors exist. For example, the segmentation model may be a model based on a U-net or a Transformer.

Alternatively, in one embodiment, based on the CT image and the information of organ delineation, the weight of a path through which a particle beam passes in the tumor and normal tissue may be adjusted.

Alternatively, in one embodiment, after setting a center of mass position in the tumor information, the distance from the surface to the center of mass position may be calculated to compute the distance information from the surface to the tumor.

The deep learning model may be trained using the processed patient data.

The training may be performed after converting the processed patient data into one-dimensional data.

After the training is completed, the derived beam angle may be compared with the actual treatment result to evaluate the model.

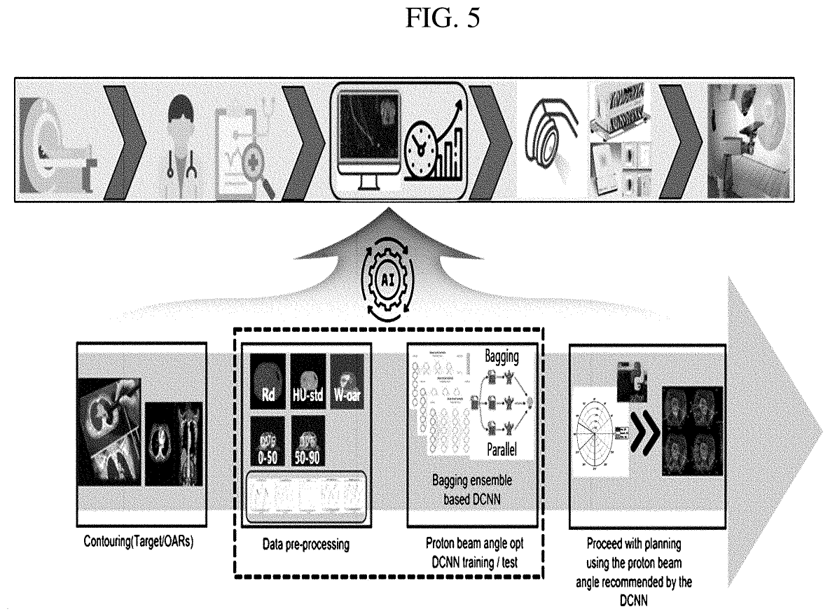

FIG. 5 illustrates one embodiment of a process of a patient-specific beam angle calculation technique for particle therapy. As shown in FIG. 5, the patient-specific beam angle calculation technique for particle therapy may include contouring medical images, performing data processing based on the contouring result, and then training or testing a deep learning model such as a DCNN (Deep Convolutional Neural Network). Subsequently, based on the deep learning model such as the DCNN, a patient-specific beam angle for particle therapy may be determined.

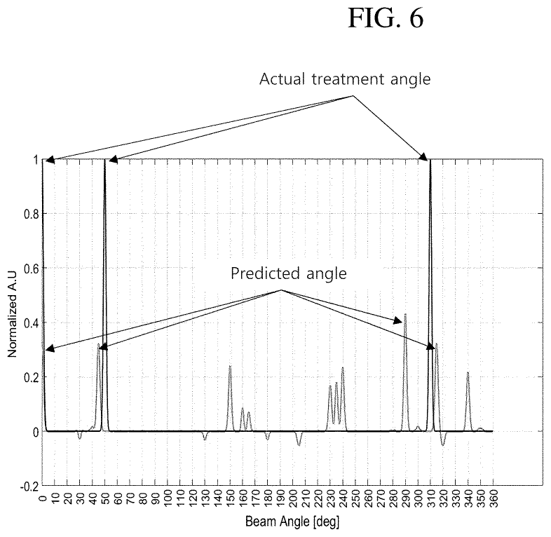

FIG. 6 shows one embodiment in which an analysis apparatus calculates a particle therapy beam angle. The analysis apparatus calculates an effect according to an angle and displays it in a graph.

It can be confirmed that, among the predicted results by the analysis apparatus, the result having a high peak substantially coincides with an actual treatment planning angle (reference data). In other words, it can be confirmed that the experimental particle therapy beam angle predicted by the analysis apparatus is reliable.

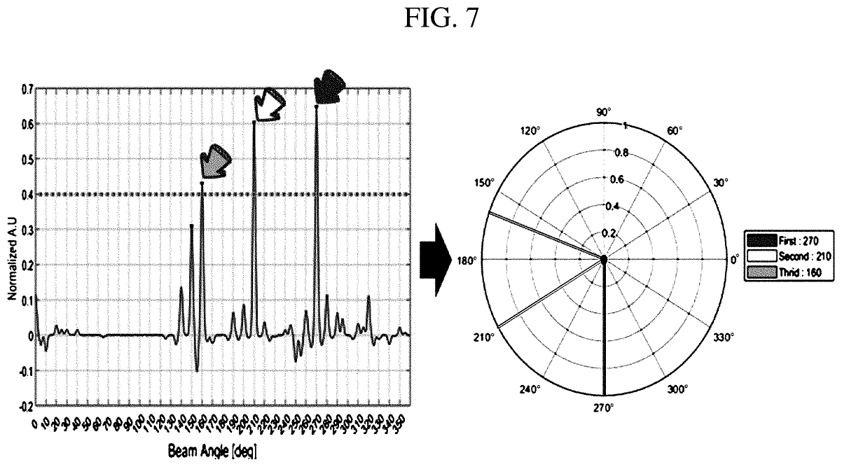

FIG. 7 shows another embodiment in which the analysis apparatus calculates a particle therapy beam angle. The analysis apparatus calculates an effect according to the beam angle and displays it as a graph. From the effect according to the predicted beam angle, the analysis apparatus may select a beam angle having an effect above a preset threshold. As shown in FIG. 7, three optimal beam angles (First, Second, and Third) may be selected. Particle therapy may be performed based on the beam angles thus selected.

Hereinafter, the analysis apparatus will be described.

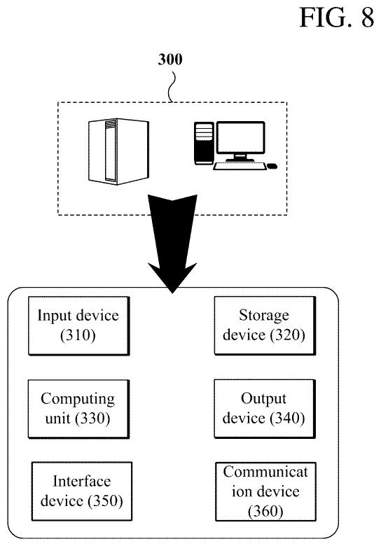

FIG. 8 shows one embodiment of a configuration of an analysis apparatus (300).

The analysis apparatus (300) may correspond to the analysis apparatus (100) described in FIG. 1. The analysis apparatus (300) may be a device that performs the aforementioned particle beam optimization method.

The analysis apparatus (300) may be physically implemented in various forms. For example, the analysis apparatus (300) may take the form of a PC, a notebook, a smart device, a server, or a data-processing dedicated chipset.

The analysis apparatus (300) may include an input device (310), a storage device (320), a computing unit (330), an output device (340), an interface device (350), and a communication device (360).

The input device (310) may include an interface device (such as a keyboard, mouse, or touchscreen) for receiving certain commands or data. The input device (310) may include a configuration for receiving information through a separate storage device (such as USB, CD, or hard disk). The input device (310) may receive data through a separate measuring device or from a separate database. The input device (310) may receive data through wired or wireless communication.

The input device (310) may receive information and a model necessary for performing the aforementioned particle beam optimization method. The input device (310) may receive distance information from a patient's body surface to a tumor, internal heterogeneity information, internal transmission information of a particle beam, and internal motion information according to the patient's respiration. The input device (310) may receive a deep learning model.

The storage device (320) may store information received through the input device (310). The storage device (320) may store information generated during a computation process performed by the computing unit (330). That is, the storage device (320) may include a memory. The storage device (320) may store results calculated by the computing unit (330).

The storage device (320) may store information and a model necessary for performing the aforementioned particle beam optimization method. The storage device (320) may store distance information from a patient's body surface to a tumor, internal heterogeneity information, internal transmission information of a particle beam, and internal motion information according to the patient's respiration.

The computing unit (330) may be a device such as a processor, an application processor (AP), or a chip in which a program is embedded, that processes data and performs certain computations. The computing unit (330) may generate a control signal that controls the analysis apparatus (300).

The computing unit (330) may perform computations necessary for performing the aforementioned particle beam optimization method. The computing unit (330) may input the distance information from a patient's body surface to a tumor, the internal heterogeneity information, the internal transmission information of a particle beam, and the internal motion information according to the patient's respiration into a deep learning model. The computing unit (330) may calculate a particle therapy beam angle based on an output value of the deep learning model.

The output device (340) may be a device for outputting certain information. The output device (340) may output interfaces necessary for data processing, input data, and analysis results. The output device (340) may be physically implemented in various forms such as a display or a document output device.

The interface device (350) may be a device for receiving certain commands and data from the outside. The interface device (350) may receive information and a model necessary for performing the aforementioned particle beam optimization method from a physically connected input device or an external storage device. The interface device (350) may receive a control signal for controlling the analysis apparatus (300). The interface device (350) may output analysis results of the analysis apparatus (300).

The communication device (360) may refer to a configuration that receives and transmits certain information through wired or wireless networks. The communication device (360) may receive control signals necessary for controlling the analysis apparatus (300). The communication device (360) may transmit analysis results of the analysis apparatus (300).

The aforementioned beam angle optimization method for particle therapy may be implemented as a program (or application) including an executable algorithm that can be executed by a computer.

The program may be provided by being stored in a temporary or non-transitory computer-readable medium.

The term “non-transitory computer-readable medium” refers to a medium that permanently stores data and can be read by a device, rather than a medium that stores data only for a short time such as a register, cache, or memory. Specifically, various applications or programs described above may be stored and provided in non-transitory computer-readable media such as a CD, DVD, hard disk, Blu-ray disk, USB, memory card, ROM (read-only memory), PROM (programmable read-only memory), EPROM (erasable PROM), EEPROM (electrically erasable PROM), or flash memory.

The term “temporary computer-readable medium” refers to various types of RAM, such as Static RAM (SRAM), Dynamic RAM (DRAM), Synchronous DRAM (SDRAM), Double Data Rate SDRAM (DDR SDRAM), Enhanced SDRAM (ESDRAM), Synclink DRAM (SLDRAM), and Direct Rambus RAM (DRRAM).

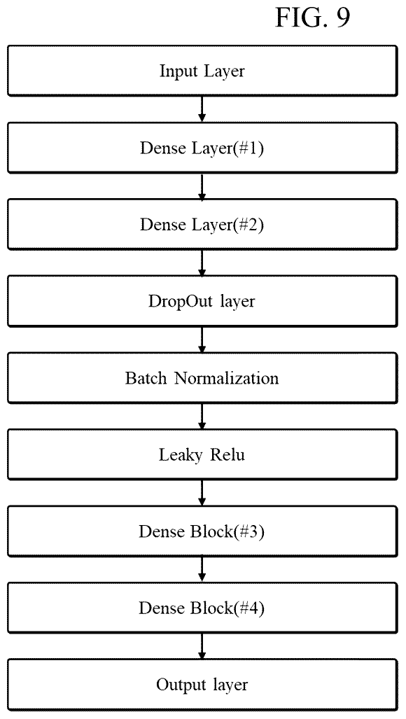

FIG. 9 shows one embodiment of a deep learning model.

The deep learning neural network may include an input layer, a feature encoding layer, a deep dense block layer, and an output layer.

The input layer may receive a plurality of three-dimensional medical image data (for example, CT, MRI, or 4D-CT) obtained from a patient's imaging information. The input layer may also receive deformation vector fields (DVF) or motion tracking parameters according to the patient's respiratory phase.

At this time, the input data may include pixel-level intensity information, HU values of tissues, spatial coordinates of structures, and segmentation masks. The spatial relationship between a tumor (PTV) and critical organs (OAR) is maintained and provided to the neural network.

The feature encoding layer may include a plurality of dense layers and batch normalization layers. The feature encoding layer converts spatial and geometric features from each input datum into a compressed vector form. The input is divided into three parts—anatomical information, motion information, and treatment equipment constraints (such as gantry angle and couch limit)—which are processed in parallel and then integrated through a concatenation operation.

In this process, a nonlinear activation function (LeakyReLU) is applied so that the model can learn complex inter-organ interactions and dose trade-off relationships.

The deep dense block layer is a core structure of the analytical model and is configured by sequentially connecting a plurality of dense blocks. Each dense block includes a fully connected layer, a dropout layer, and a normalization layer. The dropout layer deactivates neurons at a certain probability to prevent overfitting. In addition, the output of each block is transferred to the next block through skip or residual connections to minimize information loss, thereby preserving long-range dependencies.

In particular, in order to efficiently learn the interdependencies among beam geometries, a multi-dimensional feature embedding is applied, and in some implementations, a Transformer structure is introduced to calculate attention weights among multiple angles, thereby selecting clinically valid optimal incident angle candidates.

The output layer generates a probability distribution for possible incident angles (in the range of 0° to 360°) of the radiation beam based on the feature vector transmitted from the deep layers. The maximum value or top candidates of this probability distribution are selected as optimal beam directions to be used in the treatment plan.

Each candidate angle is expressed as a relative weight map and is provided to the dose optimization stage in a radiation treatment planning system (RTP). The network of the present disclosure is designed to be directly linked to existing TPS environments (such as Eclipse or RayStation), thereby automating the manual beam angle design process and reducing the overall planning time from several hours to less than several minutes.

The embodiments and drawings attached to this specification merely illustrate some of the technical ideas included in the above-described technology, and it is obvious that all modifications and specific embodiments that can be easily derived by those skilled in the art within the technical scope of the specification and drawings are included within the scope of the present disclosure.

What is claimed is:

1. A beam angle optimization method for particle therapy, comprising:
receiving, by an analysis apparatus, distance information from a patient's body surface to a tumor, internal heterogeneity information, internal transmission information of a particle beam, and internal motion information according to the patient's respiration;
inputting, by the analysis apparatus, the distance information from the patient's body surface to the tumor, the internal heterogeneity information, the internal transmission information of the particle beam, and the internal motion information according to the patient's respiration into a deep learning model; and
calculating, by the analysis apparatus, a particle therapy beam angle based on an output value of the deep learning model.

2. The method of claim 1, wherein the particle is a heavy Ion or a proton.

3. The method of claim 1, wherein the internal heterogeneity information includes information calculated based on a standard deviation value of a Hounsfield Unit (HU) obtained from a computed tomography (CT) image.

4. The method of claim 1, wherein the internal motion information according to the patient's respiration includes information calculated from deformation vector fields (DVF).

5. The method of claim 1, wherein the distance information from the patient's body surface to the tumor, the internal heterogeneity information, the internal transmission information of the particle beam, and the internal motion information according to the patient's respiration include information according to a particle beam angle.

6. The method of claim 1, wherein calculating the particle therapy beam angle includes computing, for gantry angles from 0° to 360°, a treatment-planning score and identifying a peak of the score

7. A beam angle optimization apparatus for particle therapy, comprising:
an input device configured to receive distance information from a patient's body surface to a tumor, internal heterogeneity information, internal transmission information of a particle beam, and internal motion information according to the patient's respiration;
a computing unit configured to input the distance information from the patient's body surface to the tumor, the internal heterogeneity information, the internal transmission information of the particle beam, and the internal motion information according to the patient's respiration into a deep learning model, and to calculate a particle therapy beam angle based on an output value of the deep learning model; and
a storage device configured to store the deep learning model.

8. The apparatus of claim 7, wherein the particle is a heavy Ion or a proton.

9. The apparatus of claim 7, wherein the internal heterogeneity information includes information calculated based on a standard deviation value of a Hounsfield Unit (HU) obtained from a computed tomography (CT) image.

10. The apparatus of claim 7, herein the internal motion information according to the patient's respiration includes information calculated from deformation vector fields (DVF).

11. The apparatus of claim 7, wherein the distance information from the patient's body surface to the tumor, the internal heterogeneity information, the internal transmission information of the particle beam, and the internal motion information according to the patient's respiration include information according to a particle beam angle.

12. The apparatus of claim 7, wherein calculating the particle therapy beam angle includes computing, for gantry angles from 0° to 360°, a treatment-planning score and identifying a peak of the score.
